(19) United States
(12) Patent Application Publication
(10) Pub. No.: US 2007/0208415 A1
(43) Pub. Date: Sep. 6, 2007

(54) Bifurcated stent with controlled drug delivery

(76) Inventors: Kevin Grotheim, Maple Grove, MN (US); Daniel Gregorich, St. Louis Park, MN (US)

Correspondence Address: VIDAS, ARRETT & STEINKRAUS, P.A., SUITE 400, 6640 SHADY OAK ROAD, EDEN PRAIRIE, MN, 55344, US

(21) Appl. No.: 11/368,932

(22) Filed: Mar. 6, 2006

(51) Int. Cl. A61F 2/90

(52) U.S. Cl. 623/1.16; 623/1.35; 623/1.42

(57) ABSTRACT

A bifurcated stent has at least one main stent body and at least one side branch when expanded. The bifurcated stent further has a drug eluting coating or coatings selectively deposited on the stent surface such that at least one region of the stent releases drug at a different kinetic rate than one or more adjacent regions of the stent surface.

### FIELD OF THE INVENTION

The present invention relates to the field of medical stents and, more particularly, to a stent for the treatment of lesions and other problems in or near a vessel bifurcation.

### BACKGROUND OF THE INVENTION

Stents, grafts, stent-grafts, vena cava filters, expandable frameworks, and similar implantable medical devices, collectively referred to hereinafter as stents, are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. Stents may be used to reinforce body vessels and to prevent restenosis following angioplasty in the vascular system. They may be self-expanding, expanded by an internal radial force, such as when mounted on a balloon, or a combination of self-expanding and balloon expandable (hybrid expandable).

Within the vasculature it is not uncommon for stenoses to form at a vessel bifurcation. A bifurcation is an area of the vasculature or other portion of the body where a first (or parent) vessel is bifurcated into two or more tubular component vessels. Where a stenotic lesion or lesions form at such a bifurcation, the lesion(s) can affect only one of the vessels (i.e., either of the tubular component vessels or the parent vessel), two of the vessels, or all three vessels.

The bifurcated stents may have a variety of configurations including, for example, segmented structures which include a primary branch and at least one secondary branch which is positioned adjacent to and/or partially within the primary branch. These segmented systems may employ multiple catheters and/or balloons to deploy all of the stent segments.

Other bifurcated stents include single structure stents wherein the stent is comprised of a trunk with two or more branches extending therefrom.

Still other stent configurations employ a single substantially tubular stent which has a specialized side-branch opening through which an additional stent or structural component may be deployed.

In combination with stent systems, it has further been found to be advantageous to employ pharmacologically active therapeutic agents, such as those in the form of a drug eluting coating, to reduce the amount of restenosis caused by intimal hyperplasia.

However, restenosis may not occur at the same rate or level in all regions of a bifurcated vessel. There remains a need in the art for a stent system in which the drug dosage can be optimized in specific, high risk restenosis regions within a bifurcated lesion.

The information described above is not intended to constitute an admission that such information referred to herein is “prior art” with respect to this invention.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### SUMMARY OF THE INVENTION

In one aspect, the present invention is directed to a medical device such as a stent for use in a bifurcated body lumen having a main branch and a side branch. The medical device has a radially expandable generally tubular body having proximal and distal opposing ends with a body wall having a surface extending therebetween.

In one embodiment, the medical device further includes a branch portion.

The branch portion may be outwardly deployable from the medical device body into the branch vessel.

The medical device further includes a coating including at least one therapeutic agent. The coating may be selectively disposed on the medical device surface such that the concentration of therapeutic agent is greater on some portions than others.

The medical device may further have portions which have no coating.

Selective disposition of the coating allows for optimal drug delivery to specific locations within a body lumen, such as a bifurcation.

In one embodiment, the coating is disposed on the surface of a bifurcated medical device so as to allow optimal drug delivery in areas of high restenosis, for example, near the ostium of a bifurcated lesion.

In another embodiment, the present invention relates to a method of treating a bifurcated lesion with the medical devices described herein. The method including the steps of mounting the medical device on a catheter, advancing the medical device through a body vessel to a site of an ostial bifurcated lesion, deploying the medical device at the ostial bifurcated lesion and retracting the catheter from the body vessel. The method may further include the steps of coating the medical device with one or more layers with one or more drug eluting coatings.

These and other aspects, embodiments and advantages of the present invention will be apparent to those of ordinary skill in the art upon review of the Detailed Description and Claims to follow.

### BRIEF DESCRIPTION OF THE DRAWINGS

FIG. 1 is a side view of a blood vessel bifurcation and an unexpanded stent mounted on an exemplary stent delivery system.

FIG. 1a is a perspective view of the stent shown in FIG. 1

FIG. 2 is a side view of a stent similar to that shown in FIG. 1 in an expanded condition within a bifurcated blood vessel.

FIG. 3 is a side view of a stent similar to that shown in FIG. 1 in an expanded condition within a bifurcated blood vessel.

FIG. 4 is a flat view of an embodiment of a bifurcated stent having a drug eluting coating according to the invention.

FIG. 5 is a flat view of a bifurcated stent similar to that shown in FIG. 4 with an alternative disposition of drug eluting coating.

FIG. 6 is a flat view of a bifurcated stent similar to that shown in FIGS. 4 and 5 with an alternative disposition of drug eluting coating on the stent surface.

FIG. 7 is a flat view of a bifurcated stent similar to that shown in FIGS. 4 and 5 with an alternative disposition of drug eluting coating on the stent surface.

### DETAILED DESCRIPTION OF THE INVENTION

While this invention may be embodied in many different forms, there are described in detail herein specific embodiments of the invention. This description is an exemplification of the principles of the invention and is not intended to limit the invention to the particular embodiments illustrated.

All published documents, including all US patent documents, mentioned anywhere in this application are hereby expressly incorporated herein by reference in their entirety. Any copending patent applications, mentioned anywhere in this application are also hereby expressly incorporated herein by reference in their entirety.

Depicted in the figures are various aspects of the invention. Elements depicted in one figure may be combined with, and/or substituted for, elements depicted in another figure as desired.

Embodiments of the present invention relate to a stent for use in a bifurcated body lumen having a main branch and a side branch. The stent includes a radially expandable generally tubular stent body having proximal and distal opposing ends with a body wall having a surface extending therebetween. The stent further includes a drug delivery coating which is selectively disposed on the surface of the stent to control the amount of drug release at specific locations within a body lumen.

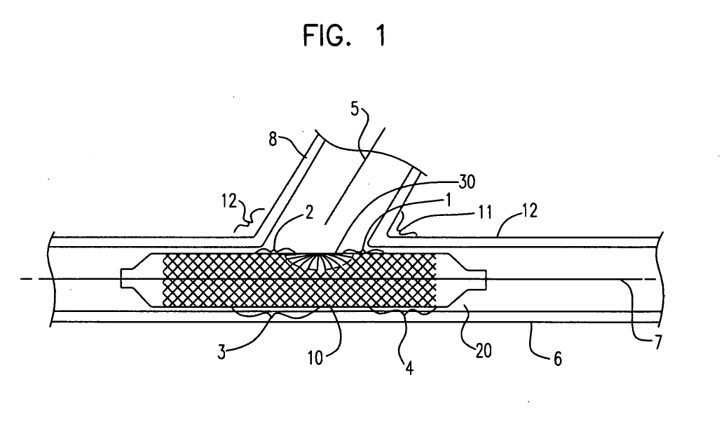

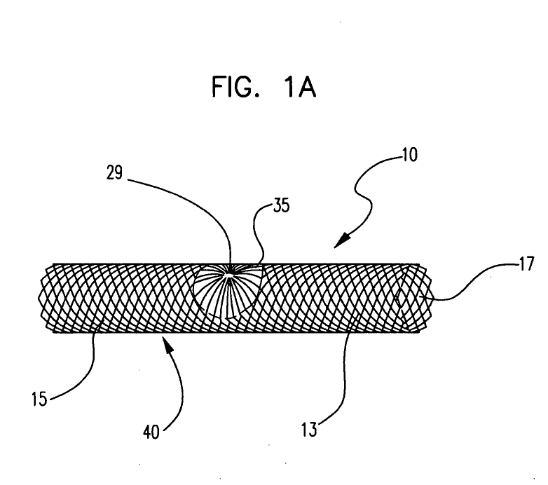

Referring to FIG. 1, for purposes of illustration only, a bifurcated blood vessel and a bifurcated stent are shown. The vessel has a main vessel 6 and a branch vessel 8. With reference to FIG. 1, bifurcated stent 10 is shown mounted on a balloon 20, and in an unexpanded configuration.

In the unexpanded state, such as is depicted in FIG. 1A, the stent 10 is shown to comprise a primary stent body 40 which itself is comprised of a plurality of interconnected stent members 13. Adjacent stent members define a plurality of openings 15 which extend through the body 40, and which are in fluid communication with the primary lumen 17 of the stent body 40.

At least one of the openings has a different shape, size, configuration, etc, than the adjacent openings 15. This different opening is a side branch opening 29, which when the stent 10 is advanced to the vessel bifurcation shown in FIG. 1, will be aligned with the ostium of the branch vessel 8.

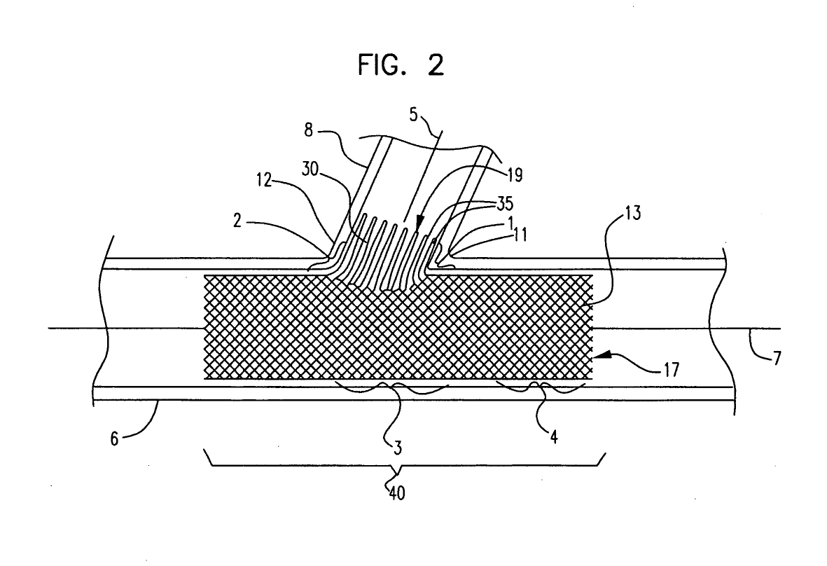

When the stent 10 is deployed or expanded at the bifurcation, such as in the manner shown in FIG. 2, one or more of the stent members 13 which surround the side branch opening will be deployed outward from the primary body 40 to form the side branch body 30. The side branch body 30 defines a side branch lumen 19 which is in fluid communication with the lumen 17 of the primary body 40. When fully deployed the longitudinal axis 5 of the side branch body 30 forms an oblique angle with the longitudinal axis 7 of the primary stent body 40.

While stent 10 depicted in FIG. 1, is shown mounted on a balloon 20, i.e. a balloon expandable stent, in some embodiments the stent 10 may include a self-expanding configuration as well.

In the embodiment shown in FIG. 2, branch 30 is shown having a plurality of finger-like projections or petals 35. The petals 35 may include any configuration of stent members 13 in order to form a branch 30 having any of a variety of desired characteristics (e.g. length, width, circumference, pattern, etc).

In the expanded state shown in FIG. 2, the side branch 30 which is, in an expanded configuration, outwardly deployed from the stent main body 40 and projecting into the branch vessel 8 of the bifurcated vessel.

In the embodiment shown above, it may be desirable to increase kinetic drug release (KDR) at or near the side branch ostium of the bifurcated lesion. This can be a high risk restenosis region. Therefore, increasing the KDR in this region may decrease the risk of restenosis. Thus, additionally, in FIGS. 1 and 2, stent 10 is shown having regions represented by reference numerals 1, 2, 3 and 4, which represent exemplary regions of stent 10 where it may be desirable to vary the drug dosage.

Increasing or decreasing the drug dosage may be accomplished in any number of ways as will be explained in detail below.

Any or all of these regions may be selected depending on the specific clinical circumstances.

Furthermore, drug eluting coating may be selectively disposed at any or all of these regions depending on specific clinical circumstances. For example at the carina, or the apex 11 of the bifurcated vessel, it may be desirable to increase the kinetic drug release in this region by twice the amount as a non-bifurcated vessel, such that both sides of the apex are effectively treated. Thus, the drug dosage at region 1 of stent 10 is increased.

Alternatively, if the highest risk region for restenosis is at the contralateral wall 12 opposite the carina 11, it may be desirable to have higher KDR in this area and consequently, higher drug dosing at region 2 on the stent surface.

Furthermore, each region may have a different level of drug eluting coating, with region 1 having the highest level, while regions, 2, 3 and 4, each respectively have less drug eluting coating.

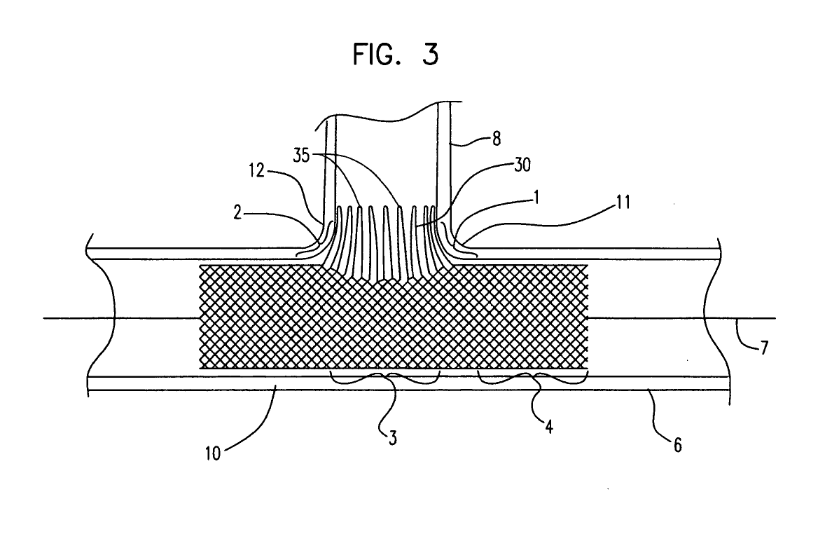

The angle of the branch may impact the selective disposition of drug eluting coating on the surface of the stent. For example, as shown in FIG. 3, wherein the branch vessel 8 is approximately perpendicular to the main branch vessel 6, it may be desirable to have equivalently higher levels of drug dosage at least in regions 1 and 2.

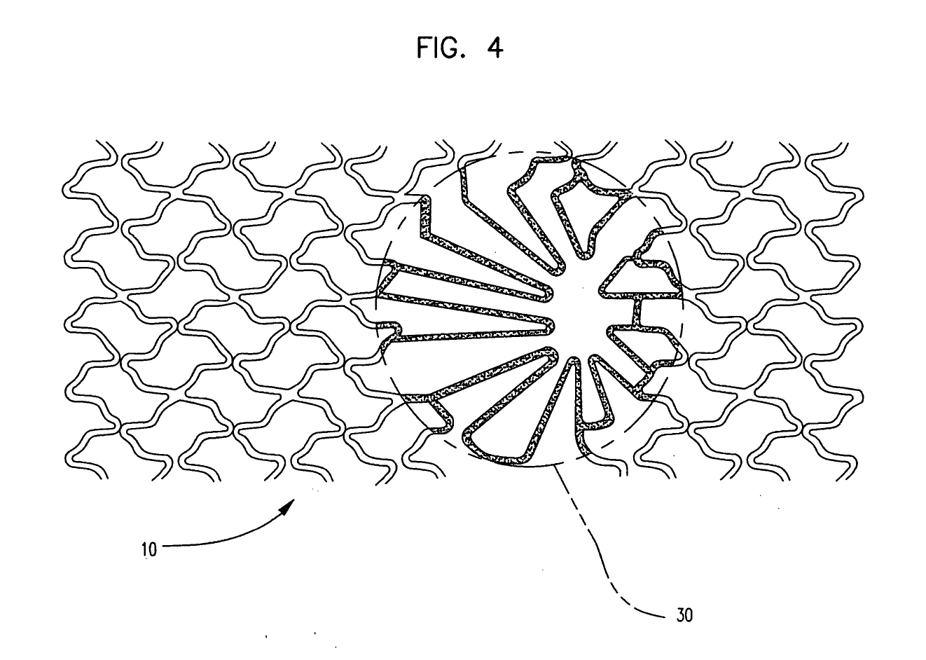

An example of an embodiment of a bifurcated stent having a drug eluting coating disposed thereon which may achieve a higher drug dosage at regions 1 and 2 (see FIG. 1). This coating disposition may obtain a higher rate of kinetic drug release at both the carina and the contraleteral wall. FIG. 4 is a flat view of a bifurcated stent 10 shown prior to expansion. In this embodiment, the stent members which eventually will make up the side branch 30 (referred to hereinafter collectively as the side branch 30), have a higher dosing of therapeutic agent then adjacent regions of the stent. This may be achieved either by increasing the coating thickness at the side branch 30, or by increasing the ratio of therapeutic agent to polymer in the coating at the side branch 30, which will be explained in more detail below.

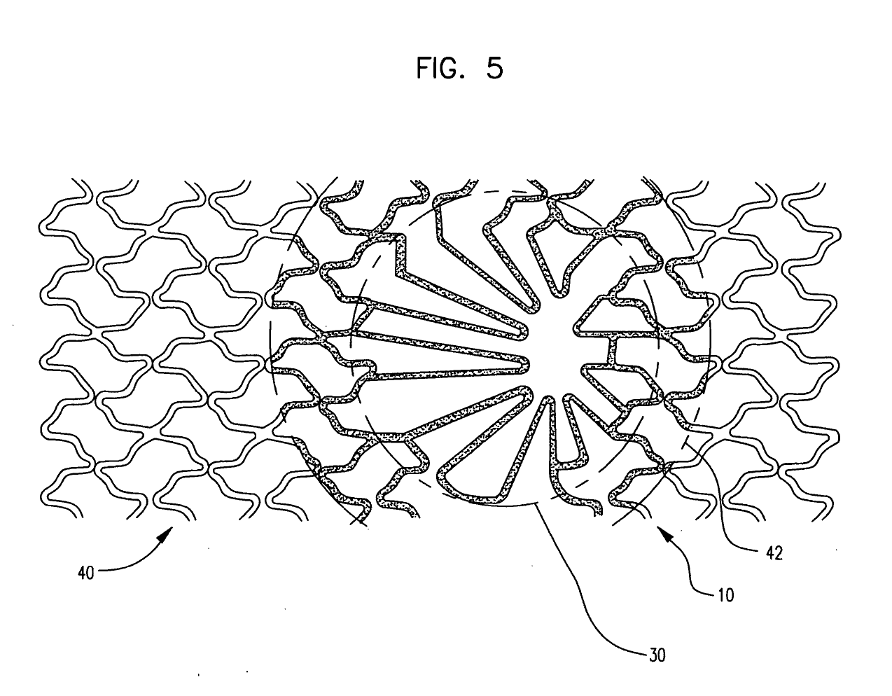

FIG. 5 is a flat view showing an alternative disposition of coating on a bifurcated stent similar to that shown in FIG. 4. In this embodiment, a higher dosing of therapeutic agent is disposed not only on entire side branch 30 of stent 10, but also on a region 42 of main stent body 40. Selective disposition of the drug eluting coating in this embodiment may more effectively increase the kinetic drug release at the ostium of a bifurcated lesion and to effectively decrease the rate of restenosis in such a location.

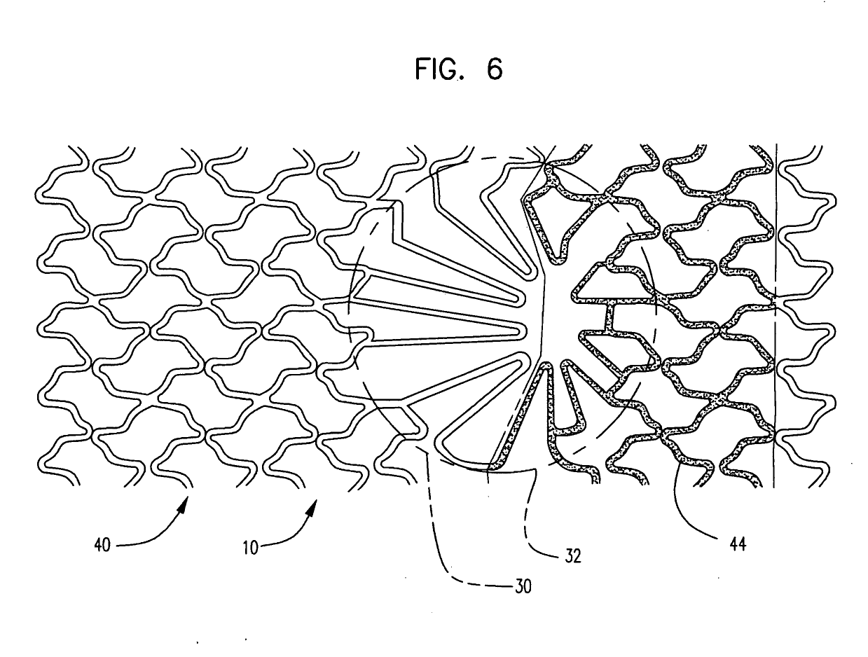

FIG. 6 is a flat view showing another embodiment coating is disposed on a region 32 of side branch 30 and a region 44 of main stent body 40 such that the drug dosing is increased in these regions of the stent. This may increase the kinetic drug release at the carina 11 as shown in FIG. 1, for example.

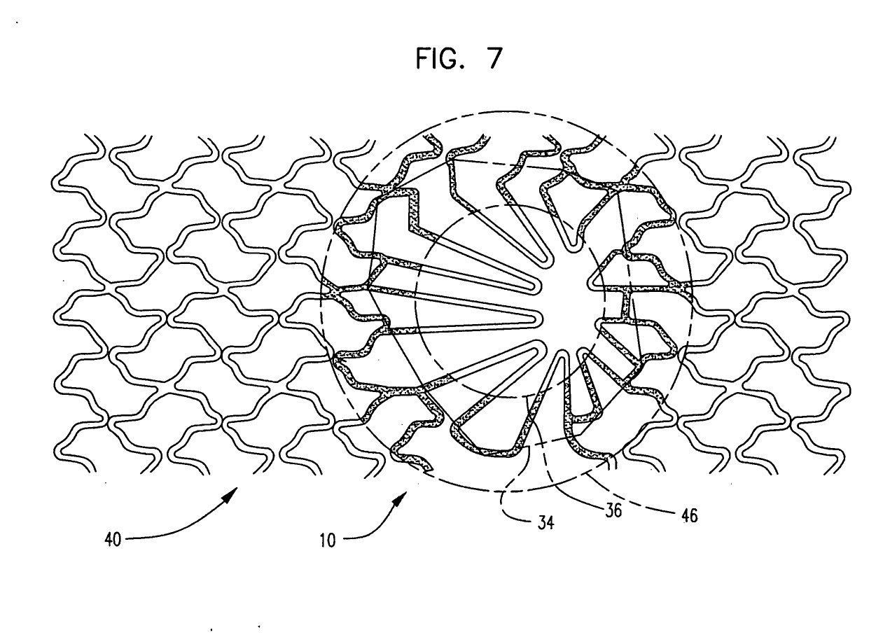

FIG. 7 is a flat view showing another embodiment wherein the coating is disposed on the surface of the stent so as to increase the drug dosing at region 34 of the side and at region 46 of main stent body 40. Region 36 of the side branch has no coating. This selective disposition of the drug eluting coating may also increase the rate of kinetic drug release at both the carina 11 and the contralateral wall 12 as shown in FIG. 1.

Furthermore, in order to increase the drug dosing at regions 3 and 4 as shown in FIGS. 1 to 3, it would be necessary to selectively place drug eluting coating on the stent surface opposite that of the side branch 30 (not shown).

The above embodiments are for purposes of illustration only, and not to limit the scope of the present invention.

While in the embodiments shown in FIGS. 4-7 show a side branch having an asymmetric crown, as described in copending U.S. Patent Publication No. US 2004/0088007, the entire content of which is incorporated by reference herein, the crown may be symmetrical as well, and may have configurations other than the finger-like projections shown. The invention is not limited by the structure of either the main body or the side branch structure of the stent.

The invention is not limited by the drug eluting coating selected. Drug eluting coatings are disclosed, for example, in commonly assigned U.S. Pat. No. 6,855,770, the entire content of which is incorporated by reference herein.

The drug eluting coating according to the invention may include at least one polymer material. Both thermoplastic and thermosetting polymer materials may be employed, as well as elastomeric and non-elastomeric polymer materials.

In some embodiments, the polymer material is a thermoplastic polymer material, and in some embodiments, the polymer material is a thermoplastic elastomer.

One suitable class of thermoplastic elastomers are styrenic block copolymers. Examples include, but are not limited to, styrene-ethylene/propylene-styrene (SEPS), styrene-butadiene-styrene (SBS), styrene-isoprene-styrene (SIS), styrene-ethylene/butylene-styrene (SEBS), styrene-isobutylene-styrene (SIBS), and so forth. Diblock copolymers of styrene and butadiene, ethylene/propylene, isoprene, ethylene/butylene, isobutylene, etc., may also be employed.

Other block copolymers which may be employed include polyamide-block-ether copolymers such as those available under the tradename of PEBAX® available from Arkema in Philadelphia, Pa., and polyester and copolyester elastomers such as poly(ester-block-ether) elastomers available under the tradename of HYTREL® from DuPont de Nemours & Co. and poly(ester-block-ester)

Other suitable polymer coating materials include, polyolefins, such as ethylene and propylene homopolymers, as well as any copolymers or terpolymers of ethylene and propylene such as ethylene-vinyl-acetate copolymers, ethylene(meth)acrylate copolymers, ethylene n-butyl acrylate copolymers, and grafted polyolefins such as maleic anhydride grafted polyethylene or polypropylene, and so forth.

Other suitable polymers which may be employed in the coatings of the invention include, but are not limited to, polyesters, polyamides including nylon 6,6 and nylon 12, polyurethanes, polyethers, polyimides, polycarboxylic acids including polyacrylic acids, (meth)acrylates, cellulosics, polycaprolactams, polyacrylamides, polycarbonates, polyacrylonitriles, polyvinylpyrrolidones, copolymers and terpolymers thereof, etc.

The coating may include bioabsorbable polymers. Examples of bioabsorbable polymers include, but are not limited to, poly(hydroxyvalerate), poly(L-lactic acid), polycaprolactone, poly(lactide-co-glycolide), poly(hydroxybutyrate), poly(hydroxybutyrate-co-valerate), polydioxanone, polyorthoesters, polyanhydrides, poly(glycolic acid), poly(D,L-lactic acid), poly(glycolic acid-co-trimethylene carbonate), polyphosphoesters, polyphosphoester urethanes, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen, hyaluronic acid, etc., and mixtures thereof.

Bioabsorbable polymers are disclosed in U.S. Pat. No. 6,790,228, the entire content of which is incorporated by reference herein.

The above lists are intended for illustrative purposes only, and are not intended to limit the scope of the present invention. Other materials not specifically listed herein, may be employed as well.

Therapeutic agent(s) may be incorporated into the coating material. “Therapeutic agents,” “drugs,” “pharmaceutically active agents,” “pharmaceutically active materials,” and other related terms are employed in the art interchangeably. Hereinafter, the term therapeutic agent will be employed herein. Therapeutic agents include genetic materials, non-genetic materials, and cells.

Examples of non-genetic therapeutic agents include, but are not limited to, anti-thrombogenic agents, anti-proliferative agents, anti-inflammatory agents, analgesics, antineoplastic/antiproliferative/anti-miotic agents, anesthetic agents, anti-coagulants, vascular cell growth promoters, vascular cell growth inhibitors, cholesterol-lowering agents; vasodilating agents; and agents which interfere with endogenous vascoactive mechanisms.

Genetic agents include anti-sense DNA and RNA and coding DNA, for example.

Cells may be of human origin, animal origin, or may be genetically engineered.

Examples of anti-thrombogenic agents include, but are not limited to, heparin, heparin derivatives, urokinase, and PPack (dextrophenylalanine proline arginine chloromethylketone).

Examples of anti-proliferative agents include, but are not limited to, enoxaprin, angiopeptin, or monoclonal antibodies capable of blocking smooth muscle cell proliferation, hirudin, acetylsalicylic acid, etc.

Examples of anti-inflammatory agents include steroidal and non-steroidal anti-inflammatory agents. Specific examples of steroidal anti-inflammatory agents include, but are not limited to, budesonide, dexamethasone, desonide, desoximetasone, corticosterone, cortisone, hydrocortisone, prednisolone, etc.

Specific examples of non-steroidal anti-inflammatory agents include, but are not limited to, acetylsalicylic acid (i.e. aspirin), ibuprofen, ibuproxam, indoprofen, ketoprofen, loxoprofen, miroprofen, naproxen, oxaprozin, piketoprofen, pirprofen, pranoprofen, protizinic acid, sulfasalazine, mesalamine, suprofen, tiaprofenic acid, etc.

Examples of analgesics include both narcotic and non-narcotic analgesics. Examples of narcotic analgesics include, but are not limited to, codeine, fentanyl, hydrocodone, morphine, promedol, etc.

Examples of non-narcotic analgesics include, but are not limited to, acetaminophen, acetanilide, acetylsalicylic acid, fenoprofen, loxoprofen, phenacetin, etc.

Examples of antineoplastic/antiproliferative/anti-miotic agents include, but are not limited to, paclitaxel, 5-fluorouracil, cisplatin, vinblastine, vincristine, epothilones, endostatin, angiostatin and thymidine kinase inhibitors.

Examples of anesthetic agents include, but are not limited to, lidocaine, bupivacaine, and ropivacaine, etc.

Examples of anti-coagulants include, but are not limited to, D-Phe-Pro-Arg chloromethyl keton, an RGD peptide-containing compound, heparin, antithrombin compounds, platelet receptor antagonists, anti-platelet receptor antibodies, aspirin, prostaglandin inhibitors, platelet inhibitors and tick antiplatelet peptides.

Derivatives of many of the above mentioned compounds also exist which are employed as therapeutic agents.

Of course mixtures of any of the above may also be employed.

The above lists are intended for illustrative purposes only, and not as a limitation on the scope of the present invention.

Therapeutic agents are discussed in commonly assigned U.S. Patent Application 2004/0215169, the entire content of which is incorporated by reference herein.

The polymer(s) and therapeutic agent(s) may be mixed in a solvent or cosolvent blend. The ratio of polymer to therapeutic agent may be from about 30:70 to about 99:1, more preferably about 70:30 to about 95:5. The resultant mixture in solvent or cosolvent blend may have a solids content of about 0.5% to about 10%, more typically about 1% to about 5%.

Any suitable solvent or cosolvent blend may be selected depending on the choice of polymer(s) and therapeutic agent(s). Suitable examples of solvents include, but are not limited to, toluene, xylene, tetrahydrofuran, hexanes, heptanes, etc.

The coating may be applied to the stent using any suitable method known in the art including, but not limited to, spraying, dipping, brushing, etc.

For illustrative purposes only, in an embodiment wherein a stent may be coated with a drug eluting coating having a ratio of polymer to therapeutic agent of about 90:10, it may be desirable to decrease the amount of polymer and increase the drug such that in desirable regions as described above, the ratio of polymer to therapeutic agent is about 80:20 to 85:15.

A first coating layer may be applied to substantially the entire stent surface, while a second coating layer may be applied only to those regions of the stent where a different rate of kinetic drug release is desirable. Of course, third, fourth, fifth, etc. layers may be applied as well.

Thus, the one or more layers of the same coating mixture may be applied in order to achieve higher drug dosing levels in particular regions of the stent. For example, a coating solution having a concentration of therapeutic agent of about 1 mg/mm² applied to the stent at a thickness of 20 microns, may be applied at a thickness of 40 microns in those regions wherein an increase rate of drug release is desirable.

Alternatively, coating layers of different coating mixtures may be applied to the stent surface. For example a first coating mixture may be applied to the entirety of the stent surface, and a second coating mixture applied only to those regions wherein different drug release is desirable.

Therefore, selective disposition of drug eluting coating may be achieved in a variety of ways which will be apparent to those of ordinary skill in the art from this description.

In some embodiments the stent may include one or more areas, bands, coatings, members, etc. that is (are) detectable by imaging modalities such as X-Ray, MRI, ultrasound, etc. In some embodiments at least a portion of the stent and/or adjacent assembly is at least partially radiopaque.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

1. A stent having an unexpanded state and an expanded state, the stent comprising:
a substantially tubular primary stent body, the primary stent body defining a primary lumen and having a longitudinal axis therethrough, the primary stent body having a surface and being comprised of a plurality of interconnected stent members, adjacent stent members defining a plurality of openings through the primary stent body, each of the openings in fluid communication with the primary lumen, at least one of the openings comprising a side branch opening, the side branch opening having a different shape than that of adjacent openings; and
a drug eluting coating, the drug eluting coating selectively deposited on the stent surface such that at least one region of the stent surface releases drug at a different kinetic rate than an adjacent region of the stent surface.

2. The stent of claim 1 wherein the adjacent stent members which define the side branch opening further define a side branch body, the side branch body defining a side branch lumen and having a longitudinal axis therethrough, in the expanded state the longitudinal axis of the side branch forming an oblique angle with the longitudinal axis of the primary stent body.

3. The bifurcated stent of claim 1, further comprising at least one second region having drug eluting coating selectively disposed thereon such that the at least one second region of the stent surface releases drug at a different kinetic rate than the at least one first region.

4. The bifurcated stent of claim 2 wherein said at least one region comprises a portion of the side branch body and a portion of the primary stent body immediately adjacent the side branch body.

5. The bifurcated stent of claim 2 wherein said at least one region comprises at least a portion of the primary stent body which is positioned substantially opposite the side branch opening.

6. The bifurcated stent of claim 1 wherein the drug eluting coating is selectively deposited on the stent surface at said at least one region so that the kinetic rate is greater at the at least one region than the adjacent region.

7. The bifurcated stent of claim 1 wherein the coating comprises a bioabsorbable polymer.

8. The bifurcated stent of claim 1 wherein the coating comprises a block copolymer.

9. The bifurcated stent of claim 7 wherein said block copolymer is a block copolymer comprising styrene endblocks.

10. The bifurcated stent of claim 8 wherein the block copolymer is selected from the group consisting of block copolymers of styrene and at least one member selected from the group consisting of ethylene/propylene, butadiene, isoprene, ethylene/butylene and isobutylene, and mixtures thereof.

11. The bifurcated stent of claim 1 wherein the therapeutic agent is paclitaxel.

12. The bifurcated stent of claim 1 wherein the stent is self-expanding.

13. The bifurcated stent of claim 1 wherein the stent is balloon expandable.

14. The bifurcated stent of claim 1 wherein the drug eluting coating comprises at least one polymer and at least one therapeutic agent, the ratio of polymer to therapeutic agent in said adjacent regions is about 90:10 to about 99:1.

15. The bifurcated stent of claim 14 wherein the drug eluting coating comprises at least one polymer and at least one therapeutic agent, the concentration of therapeutic agent in said at least one first region being approximately twice that as in said adjacent regions.

16. The bifurcated stent of claim 14 wherein the drug eluting coating is applied at a first coating thickness in the regions adjacent the at least one first region, and at a second coating thickness in said at least one first region, the coating thickness in the at least one first region is higher than the coating thickness in the regions adjacent the at least one first region.

17. A stent having an unexpanded state and an expanded state, the stent comprising:
a substantially tubular primary stent body, the primary stent body defining a primary lumen and having a longitudinal axis therethrough, the primary stent body having a surface and being comprised of a plurality of interconnected stent members, adjacent stent members defining a plurality of openings through the primary stent body, each of the openings in fluid communication with the primary lumen, at least one of the openings comprising a side branch opening, the side branch opening having a different shape than that of adjacent openings, in the expanded state adjacent stent members which define the side branch opening further define a side branch body, the side branch body defining a side branch lumen and having a longitudinal axis therethrough, in the expanded state the longitudinal axis of the side branch forming an oblique angle with the longitudinal axis of the primary body; and
a drug eluting coating, the drug eluting coating selectively deposited on at least one first region of the stent surface and at least one second region of the stent surface, wherein the at least one region of the stent surface releases drug at a greater kinetic rate than the at least one second surface

18. The bifurcated stent of claim 17, wherein the at least one first region comprises at least a portion of said side branch body and at least a portion of said primary stent body adjacent the side branch opening.

19. The bifurcated stent of claim 17 wherein the at least one first region comprises at least a portion of said side branch body and at least a portion of said primary stent body positioned substantially opposite to said side branch opening.

20. A method of treating a vessel bifurcation with a medical device, the method comprising the steps of:
mounting said medical device on a catheter, the medical device comprising at least one primary body and at least one side branch body, the medical device further comprising a first drug eluting coating, the first drug eluting coating selectively deposited on the surface of the medical device such that at least one region of said medical device releases drug at a greater kinetic rate than adjacent regions of the stent surface; and
advancing said medical device through a body vessel to a site of an ostial bifurcation lesion;
deploying said medical device at said ostial bifurcation lesion; and
retracting said catheter from said body vessel.
